SEQUENCE LISTING (4 sequences)

SEQ ID NO: 1 - DNA, 531 bp, Artificial  sequence
  Synthesized
atgcctagatcaacggaccctcttgttattggaggtgtaataggagatgttttggagcctttcacaagtagcgtttctatggggatagtctataataattgccctcaagttatcaactgctgtgagctcaaaccctccaaaatcctcaatcgaccaagaattgaaattggtggagatgacctcaggaccttttacaccctggttatggtggatcctgatgcacctagcccaggcaatccaacccagagagaatatttgcactggttgataaccaatattcctgcaactacaggggcaaacttcggagaagagattgtctcctatgaaagtccacgtccaatagtagggattcatcgaatagtttttgtgttatttcgtcagctgcgtagactaactctgcaacctccaggctggcgccagaatttcaacactagagactttgctgagatttataatcttggattaccagtagcggccatgtacttcaactgtaaacgagaaaatgatcaaagcagtggaagaagaagataa

SEQ ID NO: 2 - DNA, 531 bp, Artificial  sequence
  Synthesized
atgcctagtggaagtagggatcctctcgttgttgggggagtaattggggatgtattggatccttttgaatattctattcctatgagggttacctacaataacagagatgtcagcaatggatgtgaattcaaaccctcacaagttgtcaaccaaccaagggtaaatatcggtggtgatgacctcaggaacttctatactttgattgcggttgatcccgatgcacctagcccaagtgaccccaatttgagagaatacctccattggttggtgactgatatcccagcaacaacaggggctagtttcggccatgaggttgtaacatatgaaagtccaagaccaatgatggggattcatcgtttggtgtttgtgttatttcgtcaactgggtagggagaccgtgtatgcaccaggatggcgccagaatttcaacactaaagaatttgctgaactttacaaccttggattgccagttgctgctgtctatttcaacattcagagggaatctggttctggtggaaggaggttatactaa

SEQ ID NO: 3 - DNA, 24 bp, Artificial  sequence
  Synthesized
ttaatacgactcactatagggcga

SEQ ID NO: 4 - DNA, 22 bp, Artificial  sequence
  Synthesized
agatggtgcacgatgcacagtt

(19) United States
(12) Patent Application Publication
(10) Pub. No.: US 2024/0167017 A1
(43) Pub. Date: May 23, 2024

(54) LIQUID YEAST ONE- AND TWO-HYBRID HIGH-THROUGHPUT LIBRARY SCREENING METHOD AND USE THEREOF

(71) Applicant: INSTITUTE OF CROP SCIENCES, CHINESE ACADEMY OF AGRICUL TURAL SCIENCES, Beijing (CN)

(72) Inventors: Bingjun JIANG, Beijing (CN); Yanping Qi, Beijing (CN); Tianfu HAN, Beijing (CN); Shi SUN, Beijing (CN); Cunxiang WU, Beijing (CN); Tingting WU, Beijing (CN); Shan YUAN, Beijing (CN)

(21) Appl. No.: 18/550,843

(22) PCT Filed: Jan. 27, 2022
(86) PCT No.: PCT/CN2022/074316

§ 371 Date: Sep. 15, 2023

(30) Foreign Application Priority Data
Mar. 18, 2021 (CN) 202110290570.9

(51) Int. Cl. C12N 15/10; C12N 1/16; C12N 15/81; C12Q 1/6806; C12Q 1/686; C12Q 1/6874

(52) U.S. Cl. CPC C12N 15/1072; C12N 1/16; C12N 15/81; C12Q 1/6806; C12Q 1/686; C12Q 1/6874; C12N 2501/999

(57) ABSTRACT

A liquid yeast one- and two-hybrid high-throughput library screening method and use thereof changes the screening of solid medium to liquid culture solution screening, avoiding a large number of plate coating work, and avoiding the tedious work of identifying single colonies one by one, which not only saves experiment time, but also reduces the cost and workload; at the same time, the present invention also combines high-throughput sequencing technology to improve the analysis throughput of yeast hybridization screening work, and can also improve the automation level of analysis, and can greatly reduce false negatives. More importantly, the present invention can solve the phenomenon of protein self-activation. By changing to liquid screening, known protein interaction information can be successfully obtained, which provides a new opportunity for protein-protein interaction research.

### RELATED APPLICATIONS

The present application is a U.S. National Phase of International Application Number PCT/CN2022/074316 filed Jan. 27, 2022, which claims priority to Chinese Application Number 202110290570.9 filed Mar. 18, 2021.

### INCORPORATION BY REFERENCE

The sequence listing provided in the file entitled PUS1222087_SQL_Revised.txt, which is an ASCII text file that was created on Sep. 11, 2023, and which comprises 2,254 bytes, is hereby incorporated by reference in its entirety.

### FIELD OF THE INVENTION

The present invention relates to the field of molecular biology, in particular to a liquid yeast one- and two-hybrid high-throughput library screening method and use thereof.

### BACKGROUND OF THE INVENTION

Yeast two-hybrid is an important experimental system mainly used to screen interacting proteins and verify protein interactions in molecular biology. The key element in this system is a transcription factor (such as Gal4), which contains a DNA-binding domain (DBD) and a transcriptional activation domain (AD). Protein A is fused to the DNA-binding domain (DBD) of the transcription factor (such as Gal4) as a bait, while protein B is fused to the transcriptional activation domain (AD) (called the prey) of the transcription factor (such as Gal4). Only when protein A and protein B interact, the DNA-binding domain (DBD) and the transcriptional activation domain (AD) will be tightly combined, thereby forming a complete functional transcription factor and activating the downstream reporter gene. Similarly, in the yeast one-hybrid system, by replacing a DNA-binding structural domain (e.g. Gal4) with a library protein-encoding gene, the transcriptional activation domain (AD) of a transcription factor (e.g. Gal4) activates a downstream reporter gene when the bait DNA sequence encounters a library protein that interacts with it, so that the protein specifically bound to the bait DNA sequence is recognized.

However, the above systems also have limitations. First of all, if the bait itself has self-activation, it often has the problem of self-activating the downstream reporter gene and cannot screen the interacting protein. A common way to solve this problem is to remove its activation domain by creating specific mutations in the bait sequence. However, this method may simultaneously remove the functional domains that determine the interaction. Secondly, because the bait protein or bait sequence may interact with endogenous proteins in yeast cells, it is easy to cause false positive results. At the same time, since the expression of fusion proteins in yeast cells is different from that in the plant body environment, it may interfere with the binding of interacting proteins, resulting in false negatives. Finally, the traditional yeast one- and two-hybrid system has extremely high time and labor costs, and requires a lot of plate-plating and monoclonal PCR identification.

In recent years, with the development of sequencing technology, a number of new systems for studying proteomics in yeast one- and two-hybrid have emerged, including Assay-based Y2H, Smart-pooling Y2H, Y2H-Seq, Stitch-Seq Y2H, and BFG-Y2H. These technologies expand the scope of screening by improving the library, combined with next-generation sequencing technology, and simplify the identification work to a certain extent, but they have high requirements on the quality of the library, and are expensive and cannot be applied to small-scale screening of laboratories; more importantly, the above methods have not been able to avoid the troubles caused by the cumbersome coating work and the self-activation of the bait. Therefore, there is an urgent need for a new yeast one- and two-hybrid technology that is simple, efficient, time-saving and labor-saving, and can solve the problem of self-activation, so as to provide more convenient services for the development of molecular experiments.

### SUMMARY OF THE INVENTION

One object of the present invention is to provide a liquid yeast one/two-hybrid high-throughput library screening method.

The present invention provides a method for liquid yeast one/two-hybrid high-throughput library screening, that is, a liquid yeast one-hybrid high-throughput library screening method or a liquid yeast two-hybrid high-throughput library screening method. The method includes the following steps, the method here is aimed at liquid yeast one/two-hybrid high-throughput screening of interacting proteins:
1) constructing a yeast one/two-hybrid library with a bait sequence or a bait protein and a yeast library;
2) culturing the yeast one/two-hybrid library in a liquid maintenance medium and a liquid screening medium, respectively, to obtain a control library and a treatment library;
   the screening pressure in the liquid screening medium is greater than or higher than that of the liquid maintenance medium;
3) sequencing the control library and the treatment library, and comparing the sequencing results of the two libraries, wherein differential fragments of the treatment library relative to the control library and the genes corresponding thereto are bait sequence candidate interacting genes (specifically the genes corresponding to the differential fragments), and the proteins encoded by the genes are the candidate interacting proteins of the bait protein.

In the above method, the liquid maintenance medium and the liquid screening medium are the following two combinations, and the screening pressure in the liquid screening medium is greater than that of the liquid maintenance medium:

the above requirement that the screening pressure in the liquid screening medium is greater than that in the liquid maintenance medium specifically means that the types of antibiotics in the liquid screening medium are more than those in the liquid maintenance medium or the concentration of antibiotics is higher than that in the liquid maintenance medium.

The maintenance medium of the present invention is mainly used for amplifying and propagating yeast libraries to be screened, while maintaining the diversity of the initial yeast library to the greatest extent. The maintenance medium can be any of the following medium or a combination thereof or a product added with appropriate stress reagents (including but not limited to AbA, kanamycin, ampicillin, etc.). The screening medium of the present invention is mainly used for screening yeast libraries to be screened. The screening medium is any of the following medium or a combination thereof or the product of adding appropriate stress reagents (including but not limited to AbA, kanamycin, ampicillin, etc.).
(1) a nutrient medium YPDA Broth;
(2) a single-deficient medium SD/-Trp Broth;
(3) a single-deficient medium SD/-Leu Broth;
(4) a single-deficient medium SD/-Ura Broth;
(5) a single-deficient medium SD/-His Broth;
(6) a two-deficient medium SD/-Leu/-Trp Broth;
(7) a three-deficient medium SD/-His/-Leu/-Trp Broth;
(8) a three-deficient medium SD/-Leu/-Trp/-Ura Broth;
(9) a four-deficient medium SD/-Ade/-His/-Leu/-Trp Broth;
(10) a four-deficient medium SD/-His/-Leu/-Trp/-Ura Broth;
(11) other media.

In the above method, the liquid screening medium can further include a self-activating inhibitor. In an embodiment of the present invention, the self-activating inhibitor is AbA.

In the above method, the yeast one/two-hybrid library constructed by bait sequence or bait protein and library is any of the following:
1) transforming a vector containing a bait protein gene or an induction sequence into the yeast library to obtain the yeast one/two-hybrid library;
2) transforming the vector containing the bait protein gene or the induction sequence into a yeast strain to obtain a bait strain, and then fusing the bait strain with the yeast library to obtain the yeast one/two-hybrid library;
3) transforming and integrating the linearized vector containing the bait protein gene or induction sequence into a yeast genome to obtain a bait strain; then transferring cDNA and related plasmids required for library construction into the bait strain to obtain the yeast one/two-hybrid library;
4) simultaneously transferring a bait vector and a library plasmid containing the bait protein gene or the induction sequence to a yeast strain to obtain the yeast one/two-hybrid library;
5) transforming the vector containing the bait protein gene or the induction sequence and a library sequence into a yeast strain to obtain the yeast one/two-hybrid library;
6) fusing the yeast library containing a vector of the bait protein gene or the induction sequence and the library sequence with a yeast library containing a prey vector to obtain the yeast one/two-hybrid library.

In the above method, in the step of transforming the vector containing the bait protein gene or the induction sequence into a yeast strain to obtain a bait strain, and then fusing the bait strain with the yeast library to obtain the yeast one/two-hybrid library, the following steps can further included before the fusion: detecting the self-activation of the bait sequence or the bait protein in the bait strain, and determining the inhibitor concentration for inhibiting the self-activation thereof (this step may or may not be included in the present invention).

In the above method, the following steps are further included before steps 2) and 3): extracting the plasmids of the control library and the treatment library, performing low-cycle count PCR amplification to obtain library fragments.

In the above method, the bait protein is a GmFT1a protein or a GmFT2a protein;

the method comprises the following steps:
1) transforming the vector containing the GmFT1a protein or GmFT2a protein gene into a yeast strain to obtain a bait strain; then fusing the bait strain with a yeast library constructed from ZigongDongdou cDNA to obtain the yeast one/two-hybrid library;
   before the fusion, culturing the bait strain in SD/-Trp/X-α-Gal media supplemented with different concentrations of AbA inhibitors, and selecting an AbA concentration that grows blue colonies as a screening concentration of the AbA inhibitors;
2) culturing the yeast one/two-hybrid library in a liquid maintenance medium and a liquid screening medium, respectively, to obtain the control library and the treatment library;
   the liquid maintenance medium is an SD/-Trp/-Leu two-deficient culture solution;
   the liquid screening medium is an SD/-Ade/-His/-Leu/-Trpfour-deficient culture solution containing the screening concentration of the AbA inhibitors;
3) extracting the plasmids of the control library and the treatment library, performing low-cycle count PCR amplification to obtain library fragments; high-throughput sequencing of the above library fragments, and comparing the sequencing results of the two libraries, wherein the differential fragment of the treatment library relative to the control library and the genes corresponding thereto are the bait sequence candidate interacting genes, and the proteins encoded by the genes are the candidate interacting proteins of the bait protein.

Another object of the present invention is to provide a liquid yeast one/two-hybrid high-throughput library screening kit.

The kit provided by the present invention includes vectors and yeasts required for the yeast one/two-hybrid library, the liquid maintenance medium, the liquid screening medium, a self-activating inhibitor in the above method and instruments or reagents required for high-throughput sequencing.

In the embodiment of the present invention, the liquid yeast one- and two-hybrid high-throughput library screening technology can be based on but not limited to the Y2H Matchmaker Gold system, and the specific steps are as follows:
(1) constructing a bait vector containing a target gene, and further transforming the yeast to obtain a bait strain;
(2) detecting the self-activation of the bait, and determining the inhibitor concentration that inhibits its self-activation; this step is to determine the concentration of the inhibitor AbA, and the subsequent library screening process is carried out according to this concentration; if the maximum AbA concentration still cannot be suppressed, then the library is screened according to the maximum AbA concentration.
(3) fusing and mating the bait strain with an initial yeast library strain to obtain a yeast mixture, which is a yeast library to be screened.
(4) placing a yeast library to be screened in a maintenance medium (which can be SD/-Trp/-Leu liquid medium, but not limited to SD/-Trp/-Leu liquid medium), and amplifying and propagating appropriately.
(5) dividing the yeast library to be screened into two parts, and centrifuging to enrich the cells, wherein one part is placed in the maintenance medium at a certain dilution (which can be SD/-Trp/-Leu liquid medium, but not limited to SD/-Trp/-Leu liquid medium), called the control library, and the other part is placed in the screening medium at the same dilution (which can be SD/-Ade/-His/-Leu/-Trp/AbA liquid medium, but not limited to SD/-Ade/-His/-Leu/-Trp/AbA liquid medium), called the treatment library; and amplifying and propagating appropriately under the same conditions for the same time.
(6) extracting plasmid DNA from the two libraries of the control library and the treatment library, respectively, amplifying the library fragments by low-cycle count PCR, and subjecting the library fragments to high-throughput sequencing.
(7) comparing and analyzing the above high-throughput sequencing results, and according to the results of the comparison and analysis, screening out the differential fragments of the treatment library relative to the control library and their corresponding genes as potential interacting proteins.

The liquid yeast one- and two-hybrid high-throughput library screening technology can also be any of the following schemes or a combination thereof:
(1) placing the yeast hybridization library to be screened in the maintenance medium and amplifying and propagating appropriately.
(2) dividing the amplified and propagated yeast hybridization library to be screened into two parts, and centrifuging to enrich the bacteria, wherein one part is placed in the maintenance medium at a certain dilution, called the control library, and the other part is placed in the screening medium at the same dilution, called the treatment library; and amplifying and propagating appropriately.
(3) extracting plasmid DNA from the two libraries of the control library and the treatment library, respectively, amplifying the library fragments by low-cycle count PCR, and subjecting the library fragments to high-throughput sequencing.
(4) setting appropriate repetition as required.
(5) comparing and analyzing the above high-throughput sequencing results, and according to the results of the comparison and analysis, screening out the differential fragments of the treatment library relative to the control library and their corresponding genes as potential interacting proteins.

The high-throughput sequencing of the present invention includes, but is not limited to, Illumina next-generation sequencing, 454 next-generation sequencing, ONT third-generation sequencing, PacBio third-generation sequencing, and the like.

The comparison analysis of the present invention can use comparison tools including but not limited to blastn, blastp, miniasm2, fasta36, hisat2, tophat, etc. to compare the sequencing results to reference gene or protein databases (including but not limited to swissprot protein database, nr protein database, nt nucleic acid database, reference genome, reference genome annotation data, etc.), and then according to the comparison results, compare the sequenced fragments of the processed library and the control library and the statistical indicators of the comparison of their corresponding genes or proteins (including but not limited to reference genes or the absolute number of sequenced data of the protein, the relative number of the sequenced data of the reference gene or protein, etc.), and screening the interacting proteins according to the difference in statistical indicators of the comparison.

The present invention aims to provide a simple and efficient liquid yeast one- and two-hybrid high-throughput library screening technology and its application in view of the high time cost and labor cost of the yeast one- and two-hybrid system for studying protein-protein interaction and DNA-protein interaction, and it is difficult to solve the problem of bait self-activation.

### BRIEF DESCRIPTION OF DRAWINGS

FIG. 1 shows the results of self-activation detection of pGBKT7-FT1a; 1: SD/-Trp; 2: SD/-Trp/X-α-gal; 3: SD/-Trp/X-α-gal/AbA(300 ng/mL); 4: SD/-Trp/X-α-gal/AbA (400 ng/mL).

FIG. 2 shows the result of the first round of PCR of the yeast plasmid obtained from the library screening of pGBKT7-FT1a; Marker: DL2000 Marker; 2: the first amplification of the plasmid extracted from the control library pGBKT7-FT1a+libraryC after two-deficient screening; 3: first amplification of the plasmid extracted from the treatment library pGBKT7-FT1a+libraryT after four-deficient screening.

FIG. 3 shows the results of the second round of PCR of the yeast plasmid obtained from the library screening of pGBKT7-FT1a; Marker: DL2000 Marker; 2-5: the second amplification of the plasmid extracted from the control library pGBKT7-FT1a+libraryC after two-deficient screening; 7-10: second amplification of the plasmid extracted from the treatment library pGBKT7-FT1a+libraryT after four-deficient screening.

FIG. 4 shows the yeast interaction between GmLOG and GmFT1a.

FIGS. 5A and 5B are the bimolecular fluorescence complementary interaction between GmLOG and GmFT1a; FIG. 5A: positive control: FIG. 5B: GmFT1a and GmLOG co-transformed into tobacco.

FIG. 6 shows the results of pGBKT7-FT2a self-activation detection; 1: SD/-Trp; 2: SD/-Trp/X-α-gal; 3: SD/-Trp/X-α-gal/AbA (300 ng/mL); 4: SD/-Trp/X-α-gal/AbA (500 ng/ml).

FIG. 7 shows the result of the first round of PCR of the yeast plasmid obtained from the library screening of pGBKT7-FT2a; Marker: DL2000Marker: 2: the first amplification of the plasmid extracted from the control library pGBKT7-FT2a+libraryC after two-deficient screening; 3: first amplification of the plasmid extracted from the treatment library pGBKT7-FT2a+libraryT after four-deficient screening.

FIG. 8 shows the result of the second round of PCR of the yeast plasmid obtained from the library screening of pGBKT7-FT2a; Marker: DL2000Marker: 2-5: the second amplification of the plasmid extracted from the control library pGBKT7-FT2a+libraryC after two-deficient screening; 7-10: second amplification of the plasmid extracted from the treatment library pGBKT7-FT2a+libraryT after four-deficient screening.

FIG. 9 shows the yeast interaction between GmFBA2 and GmFT2a mutant.

FIGS. 10A and 10B show the bimolecular fluorescent complementary interaction between GmLOG and GmFT1a; FIG. 10A: positive control; FIG. 10B: GmFT2a-mutant and GmFBA2 co-transfected into tobacco.

FIG. 11 shows the amino acid sequence comparison of GmFBA1 and GmFBA2.

### DESCRIPTION OF EMBODIMENTS

The following examples facilitate a better understanding of the present invention, but do not limit the present invention.

The experimental methods in the following examples are conventional methods unless otherwise specified.

The test materials used in the following examples, unless otherwise specified, were purchased from conventional biochemical reagent stores.

Soybean variety Zigong Dongdou is recorded in the following documents: Han Tianfu, Gai Junyi, Wang Jinling, Zhou Dongxing. Discovery of soybean flowering reversal phenomenon. 1998. Acta AgronomicaSinica(02): 168-171. The soybean variety Zigong Dongdou was sensitive to photoperiod. Hereinafter, the soybean variety Zigong Dongdou is referred to as Zigong Dongdou or ZGDD.

### Example 1, Liquid Yeast One- and Two-Hybrid High-Throughput Method for Screening Proteins that Interact with FT1a Protein

The reagents used in this example were as follows:

The tested variety: the soybean variety used to construct the soybean yeast library was the photoperiod-sensitive variety Zigong Dongdou, which was planted in a light incubator with 12 h light/12 h dark treatment at a temperature of 25° C. and a humidity of 65%.

Preparation of AbA (AureobasidinA) aureobasidinA mother solution: 1 mg of AureobasidinAAbA (AureobasidinA) (purchased from Beijing Liuhetong Economic and Trade Co., Ltd., catalog number 630466) was dissolved in 2 mL of methanol solution to a final concentration of 500 μg/mL, the working concentration was 0.1-0.5 μg/mL.

AbA was a reporter in the Matchmaker Gold yeast system, which can effectively screen positive clones.

Preparation of X-α-gal mother solution: 100 mg of chromogenic substrate X-α-gal (purchased from Beijing Liuhetong Economic and Trade Co., Ltd., catalog number 630462) was dissolved in 5 mL of dimethyl sulfoxide solvent to a final concentration of 20 mg/mL, the working concentration was 40 μg/mL.

X-α-Gal was a chromogenic substrate of yeast galactosidase (MEL1), which can directly detect GAL4-based yeast two-hybrid interaction on agar, and the yeast clones with positive interaction were blue.

Preparation of 0.9% NaCl: 0.9 g of NaCl was weighed, dissolved in 100 mL of distilled water and filtered through a 0.2 μm filter membrane to obtain 0.9% NaCl.

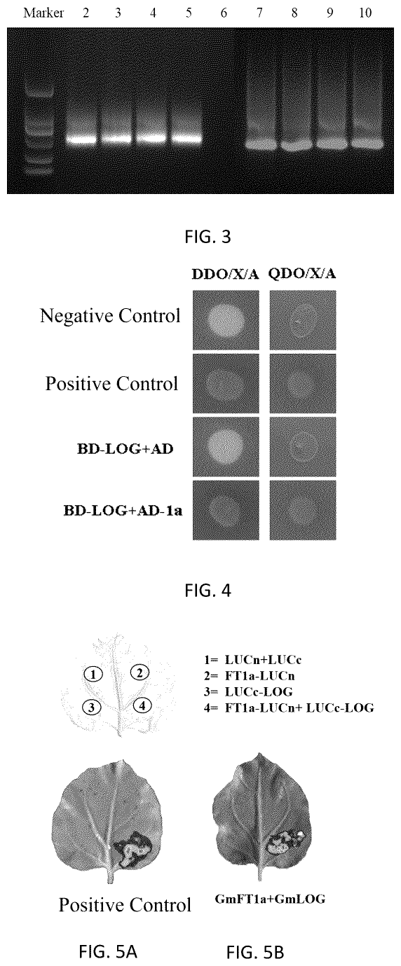

The detection process of this example: after the concentration of the AbA self-activation inhibitor was determined by pGBKT7-FT1a, the library was screened according to liquid yeast one- and two-hybrid high-throughput library screening technology. This technology was mainly aimed at improving and optimizing the processing of mating products between the library and bait yeast cells. The original two-deficient solid medium and the more stringent four-deficient solid medium were changed to corresponding liquid medium. During the library screening process, after mating with yeast, the two-deficient screening medium was added for culture; then two parts were taken out for strict two-deficient and four-deficient screening respectively, and the yeast plasmid of the final culture was extracted, and the target band was amplified by PCR It was found that after the pGBKT7-FT1a bait strain was fused with the library, the yeast plasmids of the two-deficient culture and the four-deficient culture showed a diffuse band after low-cycle count PCR amplification (FIG. 2-FIG. 3), and the band sizes ranged from 500 bp to 1000 bp, it preliminarily showed that the yeast two-hybrid technology of liquid improvement method can indeed screen candidate genes.

The details were as follows:
The soybean flowering key factor GmFT1a was used as the bait protein, the proteins interacting with GmFT1a were obtained by screening according to the liquid yeast one- and two-hybrid high-throughput library screening technology, respectively.

I. Construction of Yeast Library to be Screened

1. Acquisition of Yeast Library

When the three compound leaves of ZigongDongdou were fully expanded, the soybean leaf tissue was taken for RNA extraction, Then the library was constructed according to the kit instructions of “Make Your Own Mate & Plate Library System”, and the initial yeast library was obtained by using the pGADT7-Rec plasmid and Y187 yeast strain in the kit. 1 mL of each was subpackaged into 2 mL centrifuge tubes and stored at −80° C.

2. Fusion of the Bait Vector and the Initial Yeast Library to Obtain a Yeast Library to be Screened

1). Acquisition of the Bait Plasmid Expressing GmFT1a and the Bait Strain

the CDS sequence (sequence 1) of GmFT1a was constructed between the EcoRI and BamHI digestion sites of the pGBKT7 vector (included in the Matchmaker® Gold Yeast Two-Hybrid System kit, Clontech, 630489) to obtain the pGBKT7-FT1a bait plasmid.

The pGBKT7-FT1a bait plasmid was transferred into Y2HGold yeast competent cells (purchased from Beijing Zhuangmeng International Biogene Technology Co., Ltd, catalogue no. ZC1602) according to the kit instructions of Yeastmaker™ Yeast Transformation System 2, coated onto SD/-Trp plates and inverted cultureat 30° C. for 3 days to obtain the bait strain Y2HGold/pGBKT7-FT1a.

2) Screening of the Bait Inhibitor Concentration

The self-activation detection of the bait strain Y2HGold/pGBKT7-FT1a by different concentrations of AbA inhibitors:

Colonies of 2-3 mm in diameter were picked from the SD/-Trp culture plates of the bait strain Y2HGold/pGBKT7-FT1a obtained from 1), dissolved in 100 μL of 0.9% NaCl solution, and then 100 μL of the above solution was diluted at 1/10, 1/100, 1/1000 and coated onto nutrient-deficient plates SD/-Trp (SDO, purchased from Beijing Liuhetong Economic and Trade Co., Ltd., catalog number 630309), SD/-Trp/X-α-Gal(SDO/X was obtained by adding 20 mg/mL X-α-Gal to a final concentration of 40 μg/mL after sterilization of SDO) and SD/-Trp/X-α-Gal/AbA(SDO/X/A was obtained by adding 20 mg/mL X-α-Gal to the final concentration of 40 μg/mL after SDO sterilization, and adding 500 μg/mL AbA to the final concentration of 200 ng/ml, 300 ng/mL, 400 ng/ml, 500 ng/mL, respectively) plates containing different concentrations of AbA (AureobasidinA) respectively. The results were observed after 3 days of inverted culture at 30° C.

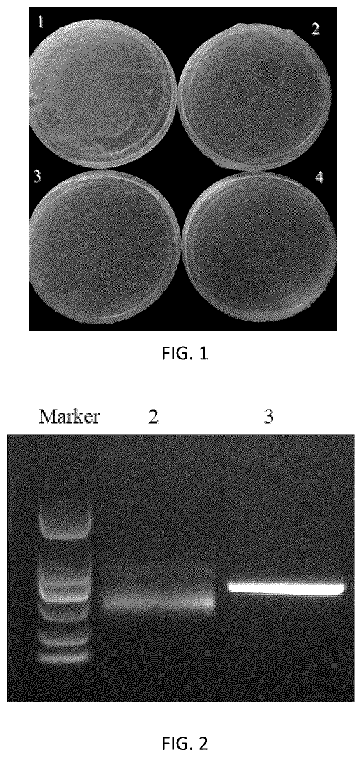

The results were shown in FIG. 1, 1-4 were the growth situation of pGBKT7-FT1a yeast in SD/-Trp, SDO/X, SDO/X/A (300 ng/mL), SDO/X/A (400 ng/mL) medium screening conditions; the bait strain Y2HGold/pGBKT7-FT1a grew blue colonies in SDO/X/A medium containing 300 ng/ml AbA(FIG. 3), indicating that GmFT1a could activate the AUR1-C gene against AbA alone. However, when AbA was 400 ng/ml, the self-activation of pGBKT7-FT1a bait strain could be inhibited and no blue colonies grew in the medium (4 in FIG. 1).

Therefore, GmFT1a was subjected to liquid yeast two-hybrid high-throughput library screening according to the AbA concentration of 400 ng/ml. This step was to determine the lowest concentration of AbA that inhibited self-activation, and then screened the library according to this concentration.

3) Concentration of Bait Strain

A large (2-3 mm) colony was picked from the SD/-Trp culture plate of the bait strain Y2HGold/pGBKT7-FT1a obtained from 1) and inoculated into 50 mL SD/-Trp liquid medium, incubated at 30° C. with shaking (250-270 rpm) until OD₆₀₀ reached 0.8 (16-20 h).

After centrifugation, the yeast cells were precipitated (1000 g, 5 min), the supernatant was discarded, and 4-5 mL SD/-Trp liquid culture solution was added to re-suspend the precipitate until the cell density>1×10⁸/mL (cells could be counted by a blood cell counter), and the concentrated bait strain Y2HGold/pGBKT7-FT1a bacteria solution was obtained.

4) Mating and Fusion

One part (1 mL) of the initial yeast library obtained from 1) above was mixed with 5 ml of the concentrated bait strain Y2HGold/pGBKT7-FT1a bacteria solution (cell density of 1.2×10⁹/mL) obtained from 3) above, and placed in a sterile 2 L flask. 45 mL of 2× YPDA liquid culture solution was added (purchased from Beijing Liuhetong Economic and Trade Co., Ltd, catalogue no. 630306, containing 50 ng/ml kanamycin) to the 2 L flask, the 2 mL centrifuge tube containing the initial yeast library was rinsed twice with 1 mL of 2× YPDA and the rinse solution was added to the 2 L flask.

Incubated at 30° ° C., 30-50 rpm for 20-24 hours until yeast zygotes appeared to obtain yeast mating products;

That is, the yeast mating product of the initial yeast library and the bait strain Y2HGold/pGBKT7-FT1a was obtained.

The lowest possible shaking speed was used in the above incubation steps to prevent cell sedimentation at the bottom of the flask; vigorous shaking may reduce mating efficiency, but too slow shaking would lead to cell sedimentation and reduce mating efficiency.

After 20 hours of incubation, a drop of the incubation product was dropped under the microscope. If a yeast zygote occurred, step 5 below was continued; otherwise, mating fusion continued and incubation continued for a further 4 hours. Zygotes usually had a 3-leaf structure, some may resemble clover leaves, while other zygotes may had a ‘Mickey Mouse’-like shape.

The above yeast mating products were transferred to 50 mL SD/-Trp/-Leu two-deficient culture solution (purchased from Beijing Liuhetong Economic and Trade Co., Ltd., catalog number 630316, containing 50 ng/mL kanamycin) and incubated at 30° C. on a shaker at 240 rpm for 2 days, the yeast library to be screened pGBKT7-FT1a+library containing the bait protein GmFT1a was obtained.

II. Identification of Proteins that Interact with the Bait Protein GmFT1a in a Liquid Yeast Two-Hybrid High-Throughput Screening Library

1. Control Library and Treatment Library

Control library: the yeast library to be screened pGBKT7-FT1a+library containing the bait protein GmFT1a obtained in the above “step 1” was centrifuged and enriched, resuspended in 10 mL 0.9% NaCl, and 5 mL was added to 45 mL SD/-Trp/-Leu two-deficient culture solution (containing 50 ng/mL kanamycin) for culturationand incubated at 30° C. on a shaker at 240 rpm for 2 days to obtain the control library pGBKT7-FT1a+libraryC.

Treatment library: From the enriched resuspension of the above yeast library to be screened pGBKT7-FT1a+library, the remaining 5 mL was added to 45 mL of SD/-Ade/-His/-Leu/-Trpfour-deficient culture solution containing 400 ng/ml of AbA (purchased from Beijing Liuhetong Economic and Trade Co., Ltd, catalogue no. 630322, containing 50 ng/ml of kanamycin) for culturation and incubated at 30° C. on a shaker at 240 rpm for 2 days to obtain the treatment library pGBKT7-FT1a+libraryT.

2. High-Throughput Sequencing

The plasmids extracted from the above control library and treatment library were amplified at low cycle counts using the universal primers of the pGADT7-Rec library plasmid (pre primer: TTAATACGACTCACTATAGGGCGA; post primer: AGATGGTGCACGATGCACAGTT) as PCR primers, and the PCR products were subjected to electrophoresis and high throughput sequencing after two rounds of PCR. The template of the second round was the low-cycle count amplification product of the first round.

The results were as follows:

FIG. 2 was the result of the first round of PCR of the yeast plasmid obtained from the screening library of pGBKT7-FT1a; Marker: DL2000 Marker; 2: the first amplification of the plasmid extracted from the control library pGBKT7-FT1a+libraryC after two-deficient screening; 3: first amplification of the plasmid extracted from the treatment library pGBKT7-FT1a+libraryT after four-deficient screening;

FIG. 3 showed the results of the second round of PCR of the yeast plasmid obtained from the screening library of pGBKT7-FT1a; Marker: DL2000 Marker; 2-5: the second round PCR products of the plasmid extracted from the control library pGBKT7-FT1a+libraryC after two-deficient screening; 7-10: the second round PCR products of the plasmid extracted from the treatment library pGBKT7-FT1a+libraryT after four-deficient screening.

It could be seen that the yeast plasmids of the control library and the treatment library showed diffuse bands after low-cycle count PCR amplification (FIG. 2-FIG. 3), with band sizes ranging from 500 bp to 1000 bp, which preliminarily showed that the yeast two-hybrid technology of liquid improvement method could indeed screen candidate genes.

The diffuse band of the above second round PCR product was recovered by gel and sent to the company for high-throughput sequencing, and the detection level of genes in the control library and the treatment library were compared, and a total of 37 candidate proteins interacting with GmFT1a were obtained.

Table 1 showed the sequencing results of the control library and the treatment library, wherein BC02 was the control library pGBKT7-FT1a+libraryC; BC04 was the treatment library pGBKT7-FT1a+libraryT; BC03 was the control library pGBKT7-FT2a+libraryC; BC05 was the treatment library pGBKT7-FT2a+libraryT;

The specific screening results of pGBKT7-FT1a+libraryT and pGBKT7-FT1a+libraryC were shown in Table 2 below:

TABLE 1
ONT sequencing results

| | Read | | N50 | N90 | Mean | Mean |
|---|---|---|---|---|---|---|
| Library | number | Total base | length | length | length | quality |
| BC02 | 19283465 | 6763477185 | 346 | 259 | 350 | 10.26 |
| BC03 | 13886419 | 5106148302 | 359 | 259 | 367 | 10.35 |
| BC04 | 10690906 | 6151197262 | 504 | 481 | 575 | 10.63 |
| BC05 | 15476307 | 5970920204 | 375 | 294 | 385 | 10.44 |

TABLE 2
Candidate proteins that interacted with GmFT1a screened by liquid
yeast two-hybrid high-throughput library screening technology

| Gene | GmFT1a | Arabi-symbol | Arabi-define |
|---|---|---|---|
| Glyma.01G029800 | + | ARFA1B | ADP-ribosylationfactorA1B |
| Glyma.02G035600 | +++ | ARFA1F | ADP-ribosylationfactorA1F |
| Glyma.02G290800 | + | PMSR4 | Peptidemetsulfoxidereductase4 |
| Glyma.03G120700 | ++ | ATCAMBP25 | Calmodulin(CAM)-binding |
| | | | protein of 25 kDa |
| Glyma.04G014500 | ++ | | PhotosystemII reaction center |
| | | | PsbP family protein |
| Glyma.04G167900 | + | CAB4 | light-harvestingchlorophyll- |
| | | | proteincomplexIsubunitA4 |
| Glyma.05G007100 | + | ATBCA1 | Carbonicanhydrase1 |
| Glyma.05G081200 | +++ | | senescence-associated protein, |
| | | | putative |
| Glyma.05G175600 | ++ | ATHB16 | Homeoboxprotein 16 |
| Glyma.06G207800 | + | | AP2/B3- |
| | | | liketranscriptionalfactorfamily |
| | | | protein |
| Glyma.07G091800 | + | | |
| Glyma.07G149300 | + | RUS5 | Protein of unknown function, |
| | | | DUF647 |
| Glyma.07G273700 | + | | |
| Glyma.08G132800 | + | ATHB16 | Homeoboxprotein 16 |
| Glyma.08G138200 | + | ATMIPS3 | myo-inositol-1- |
| | | | phosphatesynthase3 |
| Glyma.10G042000 | + | PSAE-2 | PhotosystemIsubunitE-2 |
| Glyma.10G201000 | + | LTA3 | Dihydrolipoamide |
| | | | acetyltransferase, longform |
| | | | protein |
| Glyma.11G082300 | ++ | ATSEN1 | Rhodanese/Cell cycle control |
| | | | phosphatase superfamily |
| | | | protein |
| Glyma.12G102800 | ++ | OTP82 | Tetratrico peptide repeat |
| | | | (TPR)-like superfamily protein |
| Glyma.13G012100 | +++ | | |
| Glyma.13G013900 | + | | |
| Glyma.13G021400 | + | | |
| Glyma.13G021700 | +++ | | senescence-associated protein, |
| | | | putative |
| Glyma.13G024100 | +++ | | senescence-associated protein |
| Glyma.13G140900 | +++ | LOG3 | Lonely Guy family |
| Glyma.14G150600 | +++ | ATCAO | Pheophorbidea oxygenase |
| | | | family protein with Rieske |
| | | | [2Fe—2S] domain |
| GmFT2a | + | FT | PEBP(phosphatidylethanolamine- |
| | | | binding protein) family |
| | | | protein |
| Glyma.17G040600 | + | | |
| Glyma.18G062000 | +++ | FAD8 | Fattyaciddesaturase8 |
| Glyma.18G150000 | + | SHM1 | Serinetranshydroxymethyltransferase1 |
| Glyma.18G187200 | + | CPUORF7 | Conservedpeptideupstreamope |
| | | | nreadingframe7 |
| Glyma.18G216000 | + | UBC8 | Ubiquitinconjugatingenzyme8 |
| Glyma.18G229600 | + | LSM6A | Small nuclear ribonucleo |
| | | | protein family protein |
| GmFT1a | +++ | TSF | PEBP (phosphatidylethanolamine- |
| | | | binding protein) family |
| | | | protein |
| Glyma.19G247400 | + | SDG31 | SET-domain containing protein |
| | | | lysinemethyl transferase family |
| | | | protein |
| Glyma.U005800 | ++ | | |
| Glyma.U007200 | +++ | | |

In the above table, +indicated the signal strength of the gene, and the more +, the stronger the signal.

It could be seen that the liquid yeast one- and two-hybrid high-throughput library screening technology had screened candidate proteins that interacted with GmFT1a.

The gene sequence number of GmLOG with the candidate gene number Glyma. 13 g140900 screened in the above Table 2 (https://www.soybase.org/sitemap.php) was cloned and its interoperability with GmFT1a was verified by yeast conversion experiments (validation of interactions in yeast according to the Matchmaker® Gold Yeast Two-Hybrid System) and bimolecular fluorescence complementation experiment (Yuan M,Xu Chunjue.2018.Tobacco Systems BiFC.Bio-101: e1010133).

The results of yeast conversion verification were shown in FIG. 4, DDO/X/A meant SD/-Trp/-Leu/X-α-Gal/AbA, QDO/X/A meant SD/-Ade/-His/-Leu/-Trp/X-α-Gal/AbA, BD−LOG+AD meant the strain containing BD-LOG vector and AD vector, BD−LOG+AD-1a meant the strain containing BD-LOG vector and AD-1a vector, GmLOG and GmFT1a were respectively constructed to pGBKT7 and pGADT7 vectors (included in the Matchmaker® Gold Yeast Two-Hybrid System kit, Clontech, 630489), blue colonies grew in the four-deficient SD/-Ade/-His/-Leu/-Trp after the two were co-transformed into Y2HGold yeast competent, which proved that the two did interacted;

The results of bimolecular fluorescence complementation experiment were shown in FIGS. 5A and 5B. A: positive control: B: GmFT1a and GmLOG were co-transformed into tobacco; both of them could detect obvious fluorescence signals after transient expression in tobacco; in summary, GmLOG with gene number Glyma. 13 g140900 interacted with GmFT1a.

GmLOG belonged to the LOG gene family and encoded 5′-ribose monophosphate hydrolase, which directly converted the precursor of cytokinin into cytokinin with physiological activity, and played an important role in the synthesis pathway of cytokinin. This showed that the liquid yeast one- and two-hybrid high-throughput library screening technology of the present invention was indeed feasible, and could screen positive interacting proteins in vitro interactions.

### Example 2, Liquid Yeast One- and Two-Hybrid High-Throughput Method for Screening Proteins that Interact with FT2a Protein

The reagents used in this example were as follows:

The tested variety: the soybean variety used to construct the soybean yeast library was the photoperiod-sensitive variety Zigong Dongdou, which was planted in a light incubator with 12 h light/12 h dark treatment at a temperature of 25° C. and a humidity of 65%.

Preparation of AbA (AureobasidinA) aureobasidinA mother solution: 1 mg of AureobasidinAAbA (AureobasidinA) (purchased from Beijing Liuhetong Economic and Trade Co., Ltd., catalog number 630466) was dissolved in 2 mL of methanol solution to a final concentration of 500 μg/mL, the working concentration was 0.1-0.5 μg/mL.

AbA was a reporter in the Matchmaker Gold yeast system, which can effectively screen positive clones.

Preparation of X-α-gal mother solution: 100 mg of chromogenic substrate X-α-gal (purchased from Beijing Liuhetong Economic and Trade Co., Ltd., catalog number 630462) was dissolved in 5 mL of dimethyl sulfoxide solvent to a final concentration of 20 mg/mL, the working concentration was 40 μg/mL.

X-α-Gal was a chromogenic substrate of yeast galactosidase (MEL1), which can directly detect GAL4-based yeast two-hybrid interaction on agar, and the yeast clones with positive interaction were blue.

Preparation of 0.9% NaCl: 0.9 g of NaCl was weighed, dissolved in 100 mL of distilled water and filtered through a 0.2 μm filter membrane to obtain 0.9% NaCl.

The details were as follows:

The soybean flowering key factor GmFT2a was used as the bait protein, the protein interacting with GmFT2a was obtained by screening according to the liquid yeast one- and two-hybrid high-throughput library screening technology.

I. Construction of Yeast Library Screening

1. Acquisition of Yeast Library

When the three compound leaves of Zigong Dongdou were fully expanded, the soybean leaf tissue was taken for RNA extraction, Then the library was constructed according to the kit instructions of “Make Your Own Mate & Plate Library System”, and the initial yeast library was obtained by using the pGADT7-Rec plasmid and Y187 yeast strain in the kit.

2. Construction of Yeast Library

1). Acquisition of the Bait Plasmid Expressing GmFT2a and the Bait Strain

the CDS sequence (sequence 2) of GmFT2a was constructed between the EcoRI and BamHI digestion sites of the pGBKT7 vector to obtain the pGBKT7-FT2a bait plasmid.

The pGBKT7-FT2a bait plasmid was transferred into Y2HGold yeast competent cells (purchased from Beijing Zhuangmeng International Biogene Technology Co., Ltd, catalogue no. ZC1602) according to the kit instructions of Yeastmaker™ Yeast Transformation System 2, coated onto SD/-Trp plates and inverted culture at 30° C. for 3 days to obtain the bait strain Y2HGold/pGBKT7-FT2a.

2). Screening of the Bait Inhibitor Concentration

The self-activation detection of bait strain Y2HGold/pGBKT7-FT2a by different concentrations of AbA inhibitors:

Colonies of 2-3 mm in diameter were picked from the SD/-Trp culture plates of the bait strain Y2HGold/pGBKT7-FT2a obtained from 1), dissolved in 100 μL of 0.9% NaCl solution, and then 100 μL of the above solution was diluted at 1/10, 1/100, 1/1000 and coated onto nutrient-deficient plates SD/-Trp (SDO), SD/-Trp/X-α-Gal(SDO/X) and SD/-Trp/X-α-Gal/AbA (SDO/X/A)plates containing different concentrations of AbA (AureobasidinA) (AbA concentrations were set to 200 ng/mL, 300 ng/ml, 400 ng/mL and 500 ng/ml respectively) respectively. The results were observed after 3 days of inverted culture at 30° C.

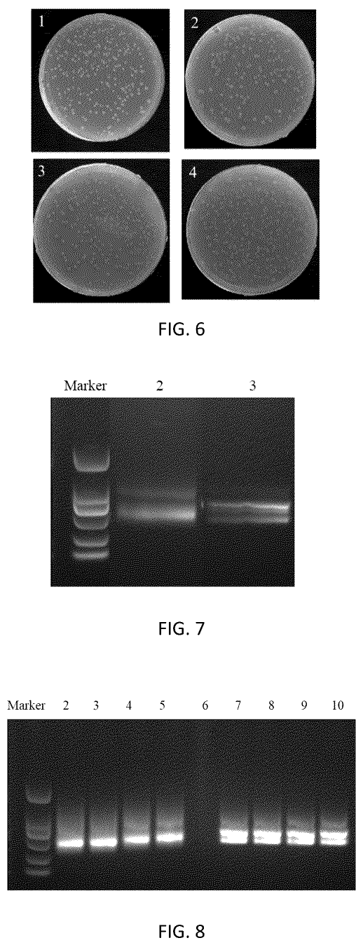

The results were shown in FIG. 6, 1-4 were the growth situation of pGBKT7-FT2a yeast in SDO, SDO/X, SDO/X/AbA (300 ng/mL), SDO/X/AbA (500 ng/mL) medium screening conditions; the bait strain Y2HGold/pGBKT7-FT2a was highly tolerant to the self-activation inhibitor AbA, and could grow colony in the medium that AbA concentration was 200 ng/ml to 500 ng/mL (3-4 in FIG. 6). This showed that GmFT2a could activate the AUR1-C gene against AbA alone, so the traditional yeast two-hybrid method cannot be used to find interacting proteins. Based on this, pGBKT7-FT2a was used as bait to verify the feasibility of liquid yeast two-hybrid high-throughput library screening technology. In order to obtain more objective sequencing results, keeping the same variables as pGBKT7-FT1a, the liquid improvement method was still used to screen the library at 400 ng/mL AbA concentration when pGBKT7-FT2a was used as bait.

3). Concentration of Bait Strain

A large (2-3 mm) colony was picked from the SD/-Trp culture plate of the bait strain Y2HGold/pGBKT7-FT2a obtained from 1) and inoculated into 50 mL SD/-Trp liquid medium respectively, incubated at 30° C. with shaking (250-270 rpm) until OD₆₀₀ reached 0.8 (16-20 h).

After centrifugation, the yeast cells were precipitated (1000 g, 5 min), the supernatant was discarded, and 4-5 mL SD/-Trp liquid culture solution was added to re-suspend the precipitate until the cell density>1×10⁸/mL (cells could be counted by a blood cell counter), and the concentrated bait strain Y2HGold/pGBKT7-FT2a bacteria solution was obtained.

4) Mating and Fusion

One part (1 mL) of the initial yeast library obtained from 1) above was mixed with 5 ml of the concentrated bait strain Y2HGold/pGBKT7-FT2a bacteria solution (cell density of 1.14×10⁹/mL) obtained from 3) above, and placed in a sterile 2 L flask. 45 mL of 2× YPDA liquid culture solution was added (purchased from Beijing Liuhetong Economic and Trade Co., Ltd, catalogue no. 630306, containing 50 ng/mL kanamycin) to the 2 L flask, the 2 mL centrifuge tube containing the initial yeast library was rinsed twice with 1 mL of 2× YPDA and the rinse solution was added to the 2 L flask.

Incubated at 30° C., 30-50 rpm for 20-24 hours until yeast zygotes appeared to obtain yeast mating products;

That is, the yeast mating product of the initial yeast library and the bait strain Y2HGold/pGBKT7-FT2a was obtained.

The lowest possible shaking speed was used in the above incubation steps to prevent cell sedimentation at the bottom of the flask; vigorous shaking may reduce mating efficiency, but too slow shaking would lead to cell sedimentation and reduce mating efficiency.

The above yeast mating products were transferred to 50 mL SD/-Trp/-Leu two-deficient culture solution (purchased from Beijing Liuhetong Economic and Trade Co., Ltd., catalog number 630316, containing 50 ng/mL kanamycin) and incubated at 30° C. on a shaker at 240 rpm for 2 days, the yeast library to be screened pGBKT7-FT2a+library containing the bait protein GmFT2a was obtained.

II. Identification of Proteins that Interact with the Bait Protein GmFT1a in a Liquid Yeast Two-Hybrid High-Throughput Screening

1. Control Library and Treatment Library

Control library: the yeast library to be screened pGBKT7-FT2a+library containing the bait protein GmFT2a obtained in the above “step 1” was centrifuged and enriched, resuspended in 10 mL 0.9% NaCl, and 5 mL was added to 45 mL SD/-Trp/-Leu two-deficient culture solution (containing 50 ng/ml kanamycin) for culturation and incubated at 30° C. on a shaker at 240 rpm for 2 days to obtain the control library pGBKT7-FT2a+libraryC.

Treatment library: From the enriched resuspension of the above yeast library to be screened pGBKT7-FT2a+library, the remaining 5 mL was added to 45 mL of SD/-Ade/-His/-Leu/-Trp four-deficient culture solution containing 400 ng/ml of AbA (containing 50 ng/ml of kanamycin) for culture and incubated at 30° C. on a shaker at 240 rpm for 2 days to obtain the treatment library pGBKT7-FT2a+libraryT.

2. High-Throughput Sequencing

The sequences from the above control library and treatment library were amplified at low cycle counts using the universal primers of the pGADT7-Rec library plasmid (pre primer: TTAATACGACTCACTATAGGGCGA(sequence 3); post primer: AGATGGTGCACGATGCACAGTT(sequence 4)) as primers, and the PCR products were subjected to electrophoresis and high throughput sequencing after two rounds of PCR.

The results were as follows:

FIG. 7 was the result of the first round of PCR of the yeast plasmid obtained from the screening library of pGBKT7-FT2a; Marker: DL2000 Marker; 2: the first amplification of the plasmid extracted from the control library pGBKT7-FT2a+libraryC after two-deficient screening; 3: first amplification of the plasmid extracted from the treatment library pGBKT7-FT2a+libraryT after four-deficient screening;

FIG. 8 showed the results of the second round of PCR of the yeast plasmid obtained from the screening library of pGBKT7-FT2a; Marker: DL2000 Marker; 2-5: the second amplification of the plasmid extracted from the control library pGBKT7-FT2a+libraryC after two-deficient screening; 7-10: the second amplification of the plasmid extracted from the treatment library pGBKT7-FT2a+libraryT after four-deficient screening.

It could be seen that, as with the results of the pGBKT7-FT1a group, the two-deficient and four-deficient cultures of pGBKT7-FT2a showed diffuse bands after low cycle count PCR amplification (FIG. 7 and FIG. 8), with band sizes ranging from 500 bp to 1000 bp, which preliminarily showed that even in the presence of self-activation of pGBKT7-FT2a, candidate proteins interacting with GmFT2a could be screened by the yeast two-hybrid technology of liquid improvement method.

The diffuse band of the above secondary amplification was recovered by gel and sent to the company for high-throughput sequencing, and the detection level of genes in the control library and the treatment library were compared, and a total of 25 candidate proteins interacting with GmFT2a were obtained (table 3).

The specific screening results of pGBKT7-FT2a+libraryT and pGBKT7-FT2a+libraryC were shown in Table 3 below:

TABLE 3
Candidate proteins that interacted with GmFT2a screened
bythe yeast two-hybrid of improvement method

| Gene | FT2a | Arabi-symbol | Arabi-define |
|---|---|---|---|
| Glyma.03G142800 | + | | Rhodanese/Cell cycle |
| | | | control phosphatase |
| | | | superfamily protein |
| Glyma.04G008300 | + | ATFBA1 | fructose- |
| | | | bisphosphatealdolase1 |
| Glyma.05G081200 | +++ | | senescence-associated |
| | | | protein, putative |
| Glyma.06G012600 | + | ATGRP7 | cold, circadian rhythm, and |
| | | | rnabinding2 |
| Glyma.06G247100 | + | PORA | Protochlorophyllide |
| | | | oxidoreductase A |
| Glyma.07G047600 | + | LHCA2 | PhotosystemIlighthar- |
| | | | vestingcomplexgene2 |
| Glyma.08G112000 | + | | ubiquitin- |
| | | | associated(UBA)/TS-N |
| | | | domain-containing protein |
| Glyma.08G300700 | + | | S-adenosyl-L-methionine- |
| | | | dependentmethyltransferase |
| | | | ssuperfamilyprotein |
| Glyma.10G043200 | + | SLT1 | HSP20- |
| | | | likechaperonessuperfamilyp |
| | | | rotein |
| Glyma.13G012100 | +++ | | |
| Glyma.13G021100 | +++ | | |
| Glyma.13G021700 | +++ | | senescence-associated |
| | | | protein, putative |
| Glyma.13G022100 | + | | senescence-associated |
| | | | protein, putative |
| Glyma.13G024100 | + | | senescence-associated |
| | | | protein |
| Glyma.13G024200 | + | | |
| Glyma.13G039100 | + | | |
| Glyma.13G263600 | + | ATMPK19 | MAPkinase19 |
| Glyma.14G032500 | + | EMB1611 | Maternaleffectembryoarrest |
| | | | 22 |
| GmFT2a | ++ | FT | PEBP(phosphatidylethanol- |
| | | | amine-binding protein) |
| | | | family protein |
| Glyma.17G002200 | + | AtHB31 | Homeoboxprotein31 |
| Glyma.18G018800 | + | | Protein of unknown |
| | | | function (DUF581) |
| Glyma.18G165300 | + | | |
| Glyma.19G051900 | +++ | RPL2.1 | RibosomalproteinL2 |
| Glyma.19G187300 | + | | |
| Glyma.U005800 | +++ | | |

In the above table, +indicated the signal strength of the gene, and the more +, the stronger the signal.

It could be seen that the protein GmFBA1 (Glyma.04G008300) protein (https://www.soybase.org/sitemap.php) interacting with GmFT2a was screened by liquid yeast one- and two-hybrid high-throughput library screening technology.

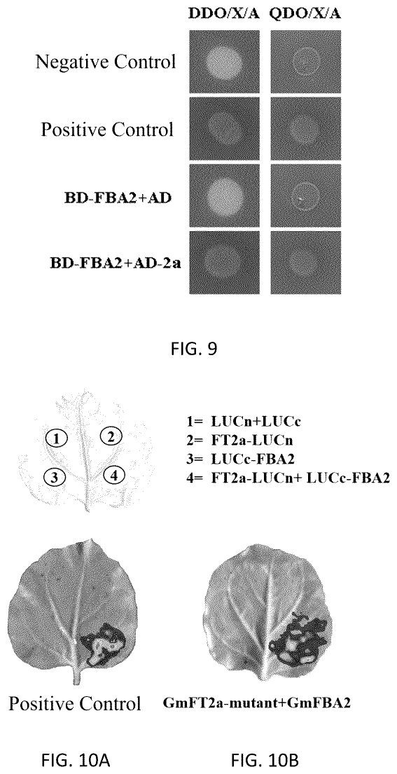

Comparative example: due to the self-activation of GmFT2a, the mutant protein GmFT2a-mutant of GmFT2a (the nucleic acid-encoded protein obtained by removing the 219-240th nucleotide sequence of GmFT2a) was obtained by removing its activation domain. GmFBA2 (Glyma.12 g037400) protein was screened by Matchmaker® Gold Yeast Two-Hybrid System yeast two-hybrid system to interact with GmFT2a-mutant(FIG. 9, DDO/X/A meant SD/-Trp/-Leu/X-α-Gal/AbA, QDO/X/A meant SD/-Ade/-His/-Leu/-Trp/X-α-Gal/AbA, BD−FBA2+AD meant strains containing BD−FBA2 vector and AD vector, BD-FBA2+AD-2a meant strains containing BD−FBA2 vector and AD-2a vector).

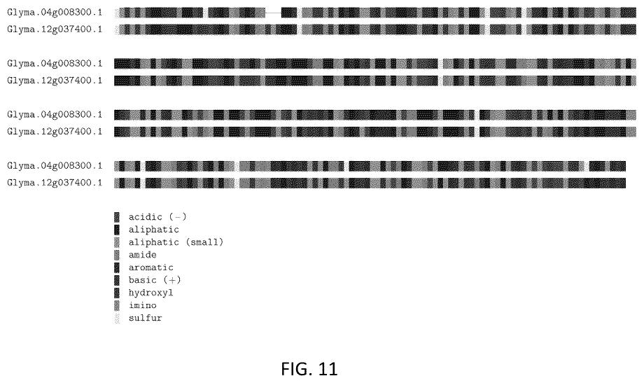

After sequence comparison, the method of the present invention screened GmFBA1 (Glyma. 04G008300) under the background of GmFT2a as the bait, which was the homologous gene of GmFBA2. Compared with the GmFBA2(Glyma.12 g037400) screened by the method of the comparative example, the similarity between the two was very high, the sequence comparison was shown in FIG. 11.

And the results of BiFC experiments showed that there was indeed an interaction between GmFT2a-mutant and GmFBA2 (FIGS. 10A and 10B), so GmFBA1 was also likely to be an interacting protein of GmFT2a.

This showed that in the case of self-activation of GmFT2a, the protein that interacted with it could still be screened by the liquid improvement method. It was further inferred that this method could avoid the problems caused by self-activation to a certain extent, and provided new ideas for the study of protein interaction, and was expected to expand the research scope of protein interaction in the future.

### INDUSTRIAL APPLICATION

On the basis of the traditional yeast hybridization system, the present invention changes the screening of solid medium to liquid culture solution screening, avoiding a large number of plate coating work, and avoiding the tedious work of identifying single colonies one by one, which not only saves experiment time, but also reduces the cost and workload; at the same time, the present invention also combines high-throughput sequencing technology to improve the analysis throughput of yeast hybridization screening work, and can also improve the automation level of analysis, and can greatly reduce false negatives. More importantly, the present invention can solve the phenomenon of protein self-activation. By changing to liquid screening, known protein interaction information can be successfully obtained, which provides a new opportunity for protein-protein interaction research. In the future, this method can find more protein-protein interactions that have not been found before due to self-activation, which provides a new opportunity for the innovation of protein-protein interaction, DNA-protein interaction and other related research methods.

1. A method for liquid yeast one/two-hybrid high-throughput library screening, comprising the steps of:
1) constructing a yeast one/two-hybrid library with a bait sequence or a bait protein and a yeast library;
2) culturing the yeast one/two-hybrid library in a liquid maintenance medium and a liquid screening medium, respectively, to obtain a control library and a treatment library;
wherein a screening pressure in the liquid screening medium is greater than or higher than that of the liquid maintenance medium;
3) sequencing the control library and the treatment library, and comparing the sequencing results of the two libraries, wherein differential fragments of the treatment library relative to the control library and genes corresponding thereto are bait sequence candidate interacting genes, and the proteins encoded by the candidate interacting genes are candidate interacting proteins of the bait protein.

2. The method according to claim 1, wherein,
the liquid maintenance medium and the liquid screening medium are the following two combinations, and the screening pressure in the liquid screening medium is greater than that of the liquid maintenance medium:
(12) a nutrient medium YPDA Broth;
(13) a single-deficient medium SD/-Trp Broth;
(14) a single-deficient medium SD/-Leu Broth;
(15) a single-deficient medium SD/-Ura Broth;
(16) a single-deficient medium SD/-His Broth;
(17) a two-deficient medium SD/-Leu/-Trp Broth;
(18) a three-deficient medium SD/-His/-Leu/-Trp Broth;
(19) a three-deficient medium SD/-Leu/-Trp/-Ura Broth;
(20) a four-deficient medium SD/-Ade/-His/-Leu/-Trp Broth;
(21) a four-deficient medium SD/-His/-Leu/-Trp/-Ura Broth;
(22) other media.

3. The method according to claim 2, wherein, the liquid screening medium further comprises a self-activating inhibitor.

4. The method according to claim 1, wherein,
a method of constructing the yeast one/two-hybrid library with the bait sequence or the bait protein and the library is any one of the followings:
1) transforming a vector containing a bait protein gene or an induction sequence into the yeast library to obtain the yeast one/two-hybrid library;
2) transforming the vector containing the bait protein gene or an induction sequence into a yeast strain to obtain a bait strain, and then fusing the bait strain with the yeast library to obtain the yeast one/two-hybrid library;
3) transforming and integrating the linearized vector containing the bait protein gene or induction sequence into a yeast genome to obtain a bait strain; then transferring cDNA and related plasmids required for library construction into the bait strain to obtain the yeast one/two-hybrid library;
4) simultaneously transferring a bait vector and a library plasmid containing the bait protein gene or the induction sequence to a yeast strain to obtain the yeast one/two-hybrid library;
5) transforming the vector containing the bait protein gene or the induction sequence and a library sequence into a yeast strain to obtain the yeast one/two-hybrid library;
6) fusing the yeast library containing a vector of the bait protein gene or the induction sequence and the library sequence with a yeast library containing a prey vector to obtain the yeast one/two-hybrid library.

5. The method according to claim 4, wherein, a method of constructing the yeast one/two-hybrid library with the bait sequence or the bait protein and the library is to transform the vector containing the bait protein gene or the induction sequence into a yeast strain to obtain a bait strain, and then fuse the bait strain with the yeast library to obtain the yeast one/two-hybrid library.

6. The method according to claim 5, wherein, the method further comprises the following steps before the fusion: detecting the self-activation of the bait sequence or the bait protein in the bait strain, and determining an inhibitor concentration for inhibiting the self-activation thereof.

7. The method according to claim 5, wherein,
the method further comprises the following steps before steps 2) and 3): extracting the plasmids of the control library and the treatment library, performing low-cycle PCR amplification to obtain library fragments.

8. The method according to claim 1, wherein,
the bait protein is a GmFT1a protein or a GmFT2a protein;
the method comprises the following steps:
1) transforming the vector containing the GmFT1a protein or GmFT2a protein gene into a yeast strain to obtain a bait strain; then fusing the bait strain with a yeast library constructed from ZigongDongdou cDNA to obtain the yeast one/two-hybrid library;
before the fusion, culturing the bait strain in SDO/-Trp/X-α-Gal media supplemented with different concentrations of AbA inhibitors, and selecting an AbA concentration that grows blue colonies as a screening concentration of the AbA inhibitors;
2) culturing the yeast one/two-hybrid library in the liquid maintenance medium and the liquid screening medium, respectively, to obtain the control library and the treatment library;
wherein the liquid maintenance medium is an SD/-Trp/-Leu two-deficient culture solution;
the liquid screening medium is an SD/-Ade/-His/-Leu/-Trpfour-deficient culture solution containing the screening concentration of the AbA inhibitors;
3) extracting the plasmids of the control library and the treatment library, performing low-cycle PCR amplification to obtain the library fragments; performing high-throughput sequencing on the above library fragments, and comparing the sequencing results of the two libraries, wherein the differential fragments of the treatment library relative to the control library and the genes corresponding thereto are the bait sequence candidate interacting genes, and the protein encoded by the candidate interacting genes are candidate interacting proteins of the bait protein.

9. A liquid yeast one/two-hybrid high-throughput library screening kit, comprising the vectors and yeasts required for the yeast one/two-hybrid library, the liquid maintenance medium, the liquid screening medium, a self-activating inhibitor in the method of claim 1 and instruments or reagents required for high-throughput sequencing.
